(19) United States
(12) Patent Application Publication
(10) Pub. No.: US 2012/0004140 A1
(43) Pub. Date: Jan. 5, 2012

(54) FLOW CELLS FOR BIOCHEMICAL ANALYSIS

(75) Inventor: Bryan P. Staker, Pleasanton, CA (US)

(73) Assignee: Complete Genomics, Inc., Mountain View, CA (US)

(21) Appl. No.: 13/232,858

(22) Filed: Sep. 14, 2011

Related U.S. Application Data
(62) Division of application No. 12/363,471, filed on Jan. 30, 2009.
(60) Provisional application No. 61/025,568, filed on Feb. 1, 2008.

(51) Int. Cl. C40B 40/06; G01N 21/75; C12M 1/40

(52) U.S. Cl. 506/16; 435/287.2; 422/82.05

(57) ABSTRACT

Assay flow cells used as part of an overall system for biological assays include, in various configurations, a carrier in which an assay substrate may be provided, where a substantial portion of the assay substrate can be used for biochemical analysis, since the carrier component of the flow cell is designed to provide functionalities that in prior art systems were performed by the assay substrate itself The flow cells may be used in automated systems, are flat for imaging and various configurations of the components of the flow cells minimize evaporation, yet allow for precise control of fluid intake and evacuation.

### CROSS-REFERENCES TO RELATED APPLICATIONS

The present application is a division of U.S. nonprovisional application Ser. No. 12/363,471 filed Jan. 30, 2009, entitled “Flow Cells for Biochemical Analysis.” This application also claims benefit under 35 USC 119(e) of U.S. provisional Application No. 61/025,568, filed on Feb. 1, 2008, entitled “Improved Flow Cells For Biochemical Analysis,” the content of which is incorporated herein by reference in its entirety.

### STATEMENT AS TO RIGHTS TO INVENTIONS MADE UNDER FEDERALLY SPONSORED RESEARCH OR DEVELOPMENT

NOT APPLICABLE

### REFERENCE TO A “SEQUENCE LISTING,” A TABLE, OR A COMPUTER PROGRAM LISTING APPENDIX SUBMITTED ON A COMPACT DISK

NOT APPLICABLE

### BACKGROUND

This invention relates to tools for biochemical analysis and in particular to a type of element used in automatic, high-throughput genome sequencing.

High-throughput analysis of chemical and/or biological species is an important tool in the fields of diagnostics and therapeutics. Arrays of attached chemical and/or biological species can be designed to define specific target sequences, analyze gene expression patterns, identify specific allelic variations, determine copy number of DNA sequences, and identify, on a genome-wide basis, binding sites for proteins (e.g., transcription factors and other regulatory molecules). In a specific example, the advent of the human genome project required that improved methods for sequencing nucleic acids, such as DNA (deoxyribonucleic acid) and RNA (ribonucleic acid), be developed. Determination of the entire 3,000,000,000 base sequence of the haploid human genome has provided a foundation for identifying the genetic basis of numerous diseases. However, a great deal of work remains to be done to identify the genetic variations associated with a statistically significant number of human genomes, and improved high throughput methods for analysis can aid greatly in this endeavor.

The high-throughput analysis approaches presently used often utilize assay devices, known as flow cells, which contain arrays of chemicals and/or biological species available for analysis. The manufacture and use of many current flow cells designs can be costly, and the flow cell design is often inefficient in the utilization of functionalized surface area, decreasing the amount of data that can be obtained using the flow cell.

### Definitions

The practice of the techniques described herein may employ, unless otherwise indicated, conventional techniques and descriptions of organic chemistry, polymer technology, molecular biology (including recombinant techniques), cell biology, biochemistry, and sequencing technology, which are within the skill of those who practice in the art. Such conventional techniques include polymer array synthesis, hybridization and ligation of polynucleotides, and detection of hybridization using a label. Specific illustrations of suitable techniques can be had by reference to the examples herein. However, other equivalent conventional procedures can, of course, also be used. Such conventional techniques and descriptions can be found in standard laboratory manuals such as Green, et al., Eds. (1999), Genome Analysis: A Laboratory Manual Series (Vols. I-IV); Weiner, Gabriel, Stephens, Eds. (2007), Genetic Variation: A Laboratory Manual; Dieffenbach, Dveksler, Eds. (2003), PCR Primer: A Laboratory Manual; Bowtell and Sambrook (2003), DNA Microarrays: A Molecular Cloning Manual; Mount (2004), Bioinformatics: Sequence and Genome Analysis; Sambrook and Russell (2006), Condensed Protocols from Molecular Cloning: A Laboratory Manual; and Sambrook and Russell (2002), Molecular Cloning: A Laboratory Manual (all from Cold Spring Harbor Laboratory Press); Stryer, L. (1995) Biochemistry (4th Ed.) W.H. Freeman, New York N.Y.; Gait, “Oligonucleotide Synthesis: A Practical Approach” 1984, IRL Press, London; Nelson and Cox (2000), Lehninger, Principles of Biochemistry 3^{rd} Ed., W. H. Freeman Pub., New York, N.Y.; and Berg et al. (2002) Biochemistry, 5^{th} Ed., W.H. Freeman Pub., New York, N.Y., all of which are herein incorporated in their entirety by reference for all purposes.

As used herein and in the appended claims, the singular forms “a,” “an,” and “the” include plural referents unless the context clearly dictates otherwise. Thus, for example, reference to “a channel” refers to one or more channels available on an assay substrate, and reference to “the method” includes reference to equivalent steps and methods known to those skilled in the art, and so forth.

Unless defined otherwise, all technical and scientific terms used herein have the same meaning as commonly understood by one of ordinary skill in the art to which this invention belongs. All publications mentioned herein are incorporated by reference for the purpose of describing and disclosing devices, formulations and methodologies that may be used in connection with the presently described invention.

Where a range of values is provided, it is understood that each intervening value, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either both of those included limits are also included in the invention.

In the following description, numerous specific details are set forth to provide a more thorough understanding of the present invention. However, it will be apparent to one of skill in the art upon reading the present disclosure that the present invention may be practiced without one or more of these specific details. In other instances, well-known features and procedures well known to those skilled in the art have not been described in order to avoid obscuring the invention.

“Amplicon” means the product of a polynucleotide amplification reaction. That is, it is a population of polynucleotides that are replicated from one or more starting sequences. Amplicons may be produced by a variety of amplification reactions, including but not limited to polymerase chain reactions (PCRs), linear polymerase reactions, nucleic acid sequence-based amplification, circle dependant amplification and like reactions (see, e.g., U.S. Pat. Nos. 4,683,195; 4,965,188; 4,683,202; 4,800,159; 5,210,015; 6,174,670; 5,399,491; 6,287,824 and 5,854,033; and U.S. Published Pat. App. No. 2006/0024711).

“Circle dependant replication” or “CDR” refers to multiple displacement amplification of a circular template using one or more primers annealing to the same strand of the circular template to generate products representing only one strand of the template. In CDR, no additional primer binding sites are generated and the amount of product increases only linearly with time. The primer(s) used may be of a random sequence (e.g., one or more random hexamers) or may have a specific sequence to select for amplification of a desired product. Without further modification of the end product, CDR often results in the creation of a linear construct having multiple copies of a strand of the circular template in tandem, i.e. a linear, single-stranded concatamer of multiple copies of a strand of the template.

“Circle dependant amplification” or “CDA” refers to multiple displacement amplification of a circular template using primers annealing to both strands of the circular template to generate products representing both strands of the template, resulting in a cascade of multiple-hybridization, primer-extension and strand-displacement events. This leads to an exponential increase in the number of primer binding sites, with a consequent exponential increase in the amount of product generated over time. The primers used may be of a random sequence (e.g., random hexamers) or may have a specific sequence to select for amplification of a desired product. CDA results in a set of concatemeric double-stranded fragments is formed.

“Ligand” as used herein refers to a molecule that may attach, covalently or noncovalently, to a molecule on an assay substrate, either directly or via a specific binding partner. Examples of ligands which can be employed by this invention include, but are not restricted to, antibodies, cell membrane receptors, monoclonal antibodies and antisera reactive with specific antigenic determinants (such as on viruses, cells or other materials), drugs, polynucleotides, nucleic acids, peptides, cofactors, lectins, sugars, polysaccharides, cells, cellular membranes, and organelles.

“Microarray” or “array” refers to a solid phase support having a surface, preferably but not exclusively a planar or substantially planar surface, which carries an array of sites containing nucleic acids such that each site of the array comprises identical copies of oligonucleotides or polynucleotides overlapping with other member sites of the array; that is, the sites are spatially discrete. The array or microarray can also comprise a non-planar interrogatable structure with a surface such as a bead or a well. The oligonucleotides or polynucleotides of the array may be covalently bound to the substrate, or may be non-covalently bound. Conventional microarray technology is reviewed in, e.g., Schena, Ed. (2000), Microarrays: A Practical Approach (IRL Press, Oxford). As used herein, “random array” or “random microarray” refers to a microarray where the identity of the oligonucleotides or polynucleotides is not discernable, at least initially, from their location but may be determined by a particular operation on the array, such as by sequencing, hybridizing decoding probes or the like. See, e.g., U.S. Pat. Nos. 6,396,995; 6,544,732; 6,401,267; and 7,070,927; WO publications WO 2006/073504 and 2005/082098; and US Pub Nos. 2007/0207482 and 2007/0087362.

“Nucleic acid” and “oligonucleotide” are used herein to mean a polymer of nucleotide monomers. As used herein, the terms may also refer to double stranded forms. Monomers making up nucleic acids and oligonucleotides are capable of specifically binding to a natural polynucleotide by way of a regular pattern of monomer-to-monomer interactions, such as Watson-Crick type of base pairing, base stacking, Hoogsteen or reverse Hoogsteen types of base pairing, or the like, to form duplex or triplex forms. Such monomers and their internucleosidic linkages may be naturally occurring or may be analogs thereof, e.g., naturally occurring or non-naturally occurring analogs. Non-naturally occurring analogs may include peptide nucleic acids, locked nucleic acids, phosphorothioate internucleosidic linkages, bases containing linking groups permitting the attachment of labels, such as fluorophores, or haptens, and the like. Whenever the use of an oligonucleotide or nucleic acid requires enzymatic processing, such as extension by a polymerase, ligation by a ligase, or the like, one of ordinary skill would understand that oligonucleotides or nucleic acids in those instances would not contain certain analogs of internucleosidic linkages, sugar moieties, or bases at any or some positions, when such analogs are incompatible with enzymatic reactions. Nucleic acids typically range in size from a few monomeric units, e.g., 5-40, when they are usually referred to as “oligonucleotides,” to several hundred thousand or more monomeric units. Whenever a nucleic acid or oligonucleotide is represented by a sequence of letters (upper or lower case), such as “ATGCCTG,” it will be understood that the nucleotides are in 5′→3′ order from left to right and that “A” denotes deoxyadenosine, “C” denotes deoxycytidine, “G” denotes deoxyguanosine, and “T” denotes thymidine, “I” denotes deoxyinosine, “U” denotes uridine, unless otherwise indicated or obvious from context. Unless otherwise noted the terminology and atom numbering conventions will follow those disclosed in Strachan and Read, Human Molecular Genetics 2 (Wiley-Liss, New York, 1999). Usually nucleic acids comprise the natural nucleosides (e.g., deoxyadenosine, deoxycytidine, deoxyguanosine, deoxythymidine for DNA or their ribose counterparts for RNA) linked by phosphodiester linkages; however, they may also comprise non-natural nucleotide analogs, e.g., modified bases, sugars, or internucleosidic linkages. It is clear to those skilled in the art that where an enzyme has specific oligonucleotide or nucleic acid substrate requirements for activity, e.g., single stranded DNA, RNA/DNA duplex, or the like, then selection of appropriate composition for the oligonucleotide or nucleic acid substrates is well within the knowledge of one of ordinary skill, especially with guidance from treatises, such as Sambrook et al, Molecular Cloning, Second Edition (Cold Spring Harbor Laboratory, New York, 1989), and like references. As used herein, “targeted nucleic acid segment” refers to a nucleic acid targeted for sequencing or re-sequencing.

“Primer” means an oligonucleotide, either natural or synthetic, which is capable, upon forming a duplex with a polynucleotide template, of acting as a point of initiation of nucleic acid synthesis and being extended from its 3′ end along the template so that an extended duplex is formed. The sequence of nucleotides added during the extension process are determined by the sequence of the template polynucleotide. Usually primers are extended by a DNA polymerase. Primers usually have a length in the range of from 9 to 40 nucleotides, or in some embodiments, from 14 to 36 nucleotides.

“Probe” as used herein refers to an oligonucleotide, either natural or synthetic, which is used to interrogate complementary sequences within a nucleic acid of unknown sequence. The hybridization of a specific probe to a target polynucleotide is indicative of the specific sequence complementary to the probe within the target polynucleotide sequence.

“Sequencing” in reference to a nucleic acid means determination of information relating to the sequence of nucleotides in the nucleic acid. Such information may include the identification or determination of partial as well as full sequence information of the nucleic acid. The sequence information may be determined with varying degrees of statistical reliability or confidence. In one aspect, the term includes the determination of the identity and ordering of a plurality of contiguous nucleotides in a nucleic acid starting from different nucleotides in the target nucleic acid.

“Substrate” is used to refer to a material or group of materials having a rigid or semi-rigid surface or surfaces. In many embodiments, at least one surface of the substrate will be substantially flat, although in some embodiments it may be desirable to physically separate synthesis regions for different compounds with, for example, wells, raised regions, pins, etched trenches, or the like. According to other embodiments, the substrate(s) will take the form of beads, resins, gels, microspheres, or other geometric configurations.

As used herein, the term “Tₘ” is used in reference to the “melting temperature.” The melting temperature is the temperature at which a population of double-stranded nucleic acid molecules becomes half dissociated into single strands. Several equations for calculating the Tₘ of nucleic acids are well known in the art. As indicated by standard references, a simple estimate of the Tm value may be calculated by the equation. Tₘ=81.5+0.41 (% G+C), when a nucleic acid is in aqueous solution at 1 M NaCl (see e.g., Anderson and Young, Quantitative Filter Hybridization, in Nucleic Acid Hybridization (1985). Other references (e.g., Allawi, H. T. & SantaLucia, J., Jr., Biochemistry 36, 10581-94 (1997)) include alternative methods of computation which take structural and environmental, as well as sequence characteristics into account for the calculation of Tₘ.

### SUMMARY

According to the invention, assay flow cells are provided that may be used as part of an overall system for chemical and/or biological assays that include, in various configurations, a carrier in which an assay substrate is positioned in a manner allowing a substantial portion of the assay substrate to be used for biochemical analysis, and specifically it may include a carrier and a coverslip with an assay substrate disposed between the carrier and the coverslip, spacers disposed defining assay regions on the assay substrate forming one or more reaction chambers with fluid inlets and outlet, between the assay substrate and the coverslip. The coverslip has a greater surface area than the assay substrate on which it is positioned so that the usable surface of the assay substrate is increased relative to conventional assay substrate-coverslip configurations. The flow cells are employed for high-speed assays. The carrier may have a recessed region capable of receiving and supporting the assay substrate and/or the coverslip.

In preferred aspects, the flow cells are used for polynucleotide analysis including, but are not limited to, nucleic acid sequencing, expression and transcriptome analysis, methylation analysis, PCR and other polynucleotide amplification reactions, SNP analysis, and the like. The flow cells of the invention are ideally suited for assays that analyze and manipulate chemical moieties, including polynucleotides, and in biological assays and/or other reactions where only small amounts of samples and reagents are employed. The flow cells of the claimed invention minimize contamination, are mechanically robust, and are easily and inexpensively manufactured. The flow cells as disclosed herein are readily used in analytic systems, including automated systems, and specific flow cell configurations can be utilized in systems with specific detection mechanisms, e.g., flow cells comprising flat surfaces are preferred for use in systems with imaging detection components. In addition, flow cells of the invention are constructed to minimize reagent evaporation, yet allow for precise control of reagent intake and evacuation.

In one implementation, the flow cell of the invention comprises: a carrier; a coverslip placed on the carrier; spacers disposed on either the carrier or the coverslip to define assay regions; and an assay substrate positioned to provide one or more reaction chambers. The coverslip is preferably thin, e.g., from approximately 100 to 500 microns in thickness, even more preferably between 150 to 300 microns in thickness, and has a greater surface area than the assay substrate on which it is positioned so that the usable surface of the assay substrate is increased relative to conventional assay substrate-coverslip configurations. The carrier may comprise one or more input and output ports, and may optionally comprise a recessed region capable of receiving and supporting the assay substrate and/or the coverslip.

The described technology provides in one aspect a flow cell with a carrier surface integrated with the sample-bearing surface of the assay substrate, which arrangement has the advantage of minimizing the number of components involved in the flow cell assembly. The flow cell of this implementation provides: a carrier comprising a bottom surface and a top surface, wherein the top surface of a region of the carrier comprises an assay substrate surface; spacers disposed on the assay substrate region of the carrier to define assay regions on the assay substrate; and a coverslip positioned on the spacers to provide one or more reaction chambers between the assay substrate and the coverslip.

In specific aspects, the coverslip is attached to both the assay substrate and the carrier, increasing the area of the coverslip that can be utilized for attachment to the flow cell. Such a configuration does not require the coverslip itself to be a mechanically robust structure, as the coverslip can be securely mounted to the carrier to provide stability to the overall structure.

In another implementation, the flow cell of the invention comprises: a carrier comprising a bottom surface and a top surface, wherein the top surface comprises a recessed region; an assay substrate comprising a top surface and a bottom surface, wherein the assay substrate is provided within the recessed region with the bottom surface of the assay substrate adjacent the recessed region; spacers disposed on the top surface of the assay substrate defining assay regions on the assay substrate; and a coverslip covering the top surface of the assay substrate.

In a specific aspect of certain implementations, the assay substrate is inset with the carrier so that the top surface of the substrate is flush with the top surface of the carrier. This allows seamless movement of fluids from the carrier surface to the assay substrate surface.

In certain aspects of these implementations, the carrier comprises an inert material such as glass, quartz, silicon, polysilicon, one or more polymers, metal, or ceramic. In other aspects, the assay substrate can comprise comprises a functionalized material, such as glass, quartz, silicon, polysilicon, or one or more polymers. The assay substrate is functionalized for specific biochemical interrogations in the assay regions. In still other aspects, the coverslip comprises an optically transmissive material such as glass, quartz, silicon, or polysilicon.

The invention also comprises systems comprising one or more flow cells of the invention and a detection device for capturing the data obtained using the flow cell. In certain implementations, the system comprises one or more imaging devices positioned adjacent to the coverslip of one or more of the flow cells.

In one specific aspect, a flow cell comprises an array of nucleic acids of known sequence arrayed in defined position on the assay substrate. In this aspect, the flow cell can comprise an array of nucleic acids with known sequence that can be used to identify binding partners, which include but are not limited to complementary nucleic acids of unknown sequence, nucleic acid binding proteins, ligands, and the like. This aspect can be useful in identification of specific binding partners in a sample, or in the determination of the presence or absence of specific sequences in a sample, using high throughput methods as described herein.

In a specific aspect, a flow cell comprises an array of target nucleic acids of unknown sequence or fragments thereof. This aspect is especially useful in the identification of a sequence of a target nucleic acid, and in particular for determination of a sequence of a complex target nucleic acid such as a complete genome using high throughput methods for determination of sequence of multiple fragments of a target nucleic acid as described herein.

In another aspect, the flow cells comprise an optional temperature control system. This is especially useful in implementations that require tightly controlled changes in temperature, e.g. amplification reactions such as PCR.

In one specific aspect of the invention, a flow cell of the invention comprises a substrate surface that has been derivatized to bind macromolecular biologic structures, e.g., amplicons of unknown sequence for high throughput, genome-scale sequencing. Derivitizing can be accomplished through a chemical reaction (e.g., addition of amine groups to one or more areas of the surface), through addition of a member of a binding pair (e.g., biotin, avidin, streptavidin and the like), through an addition of molecules with specific binding properties, e.g., oligonucleotides of known sequence, ligands, antibodies, etc. These molecules can be used to provide attachment points on the assay substrate for the molecules to be analyzed. Examples of substrates for use in flow cells of the invention include those described in more detail herein, as well as those described in U.S. Pat. Nos. 5,122,345; 5,288,468; 5,958,760; 6,326,489; 6,403,320; 6,432,360; 6,485,944; 6,511,803; 6,548,021; 6,787,308; 6,833,246; 6,960,437; 7,115,400; 7,118,910; 7,170,050; 7,220,549; 7,232,656; 7,244,559; 7,264,929; and 7,302,146; WIPO Publication WO 01/35088; and U.S. Published Patent Apps. 2006/0182664; 2007/0128610; and 2007/0172993, which are incorporated herein by reference.

In specific aspects of the invention, a flow cell designed for specific use has polymers synthesized directly on the assay substrate. Examples include, but are not limited to, those disclosed in and U.S. Published Patent Apps. 2006/0182664 and 2007/0172993, which are incorporated herein by reference.

In certain specific aspects of the invention, the flow cells may comprise a fluid port connected to a device (e.g., a syringe pump) with the ability to effect exit or entry of fluid from the flow cell. In certain implementations, the system comprises a flow cell connected to a vacuum chuck and the carrier further comprises apertures in the bottom surface of the carrier.

In another specific aspect of the invention, the flow cell comprises a port connected to a mixing chamber, which is optionally equipped with a liquid level sensor. Solutions needed for the sequencing reaction are dispensed into the chamber, mixed if needed, then drawn into the flow cell. In a preferred aspect, the chamber is conical in nature and acts as a funnel. In certain aspects of the embodiments of the invention, each flow cell comprises a temperature control subsystem with ability to maintain temperature in the range from about 5-95° C., or more specifically 10-85° C., and can change temperature with a rate of about 0.5-2° C. per second.

This Summary is provided to introduce a selection of concepts in a simplified form that are further described below in the Detailed Description. This Summary is not intended to identify key or essential features of the claimed subject matter, nor is it intended to be used to limit the scope of the claimed subject matter. Other features, details, utilities, and advantages of the claimed subject matter will be apparent from the following written Detailed Description including those aspects illustrated in the accompanying drawings and defined in the appended claims.

### BRIEF DESCRIPTION OF THE DRAWINGS

FIG. 1A is an isometric view of one aspect of a carrier according to the claimed invention.

FIG. 1B is an isometric view of one aspect of a carrier with an assay substrate in place according to the claimed invention.

FIG. 1C is an isometric view of one aspect of a coverslip positioned above the carrier according to the claimed invention.

FIG. 2A is an isometric view of one aspect of a carrier according to the claimed invention.

FIG. 2B is an isometric view of one aspect of a carrier with an assay substrate in place according to the claimed invention.

FIG. 2C is an isometric view of one aspect of a coverslip positioned on the carrier according to the claimed invention.

FIG. 3A is a top view of an assay substrate according to one aspect of the claimed invention.

FIG. 3B is a cross section of a flow cell comprising an assay substrate, carrier and coverslip according to one aspect of a flow cell of the claimed invention.

FIG. 4A is a top plan view of a carrier and a coverslip.

FIGS. 4B and 4C are cross-sectional views of three different aspects of the claimed invention.

FIGS. 5A through 5D are side plan views of various exemplary flow cells in use with a microscope.

FIGS. 6A through 6B shows a flow cell according to yet another aspect of the claimed invention.

FIGS. 7A and 7B show flow cells according to yet another aspect of the claimed invention in use with a microscope.

FIG. 8 is a side plan view of an exemplary flow cell.

### DETAILED DESCRIPTION OF THE INVENTION

The flow cells of the claimed invention include, in various configurations, a carrier in which an assay substrate is provided, where a substantial portion of the assay substrate can be used for biochemical analysis. The carrier component of the flow cell in a preferred aspect is designed to provide functionalities that in prior art systems were performed by the assay substrate itself. Such functionalities include providing fluid input and output ports, providing sufficient area for coverslip attachment, and providing areas for use for transferring the assay substrate to different system elements during processing. The flow cells of the present invention are simple, robust, and inexpensive to produce, and may be readily customized for use in a variety of applications.

In one preferred aspect, the bottom surface of the assay substrate is adjacent to a recessed region in the top surface of the carrier, and even more preferably the top surface of the assay substrate is flush with the top surface of the carrier adjacent to the recessed region. In other aspects, the bottom surface of the assay substrate sits on the surface of the carrier in a depressed region of the carrier, with the top surface of the carrier remaining above the top surface of the assay substrate. In such an aspect, the input and output ports may be provided in the ends of the carrier and applied directed to the substrate surface via channels in the carrier. Preferably, in such an aspect, the bottom side of the coverslip is flush with the carrier, and the channel height is provided by the recessed area between the top surface of the assay substrate and the bottom surface of the coverslip. In yet other aspects, the carrier itself comprises a region that acts as the substrate surface, so that these components are structurally integrated into a single unit, and the input and output ports are structurally provided by the carrier.

In other preferred aspects, the coverslip itself is utilized as the carrier, and the flow cell is configured so that the input port and output port of the flow cell are created on the coverslip. In this aspect, the detection of the assays using the substrate surface is generally detected through the coverslip, and so preferably the coverslip is composed of an optically transmissive material.

The flow cells of the invention are suited for biological assays and/or other reactions where only small amounts of samples and reagents are employed. In addition, the flow cells of the claimed invention minimize contamination, are mechanically robust, and are easily and inexpensively manufactured. The flow cells readily may be used in automated systems, as they are amenable to robotic placement (e.g., kinematic placement and positioning). The flow cells in certain aspects are flat for imaging. Additionally, various configurations of the components of the flow cells minimize evaporation yet allow for precise control of fluid intake and evacuation.

Signal detection and analysis are also facilitated in assays using the flow cells of the claimed invention since the optical properties of the components of the flow cells can be selected for specific detection systems utilizing optics and/or adjusted to incorporate features such as optical wave guides, diffraction gratings, mirrors or other optical multipliers. Thus, biological samples may be optically excited and signals may be directly detected through the flow cells, e.g., by means of a diode laser, total internal reflection MR, surface plasmon resonance (SPR), CCD or other detectors.

The structure of a flow cell typically comprises an aggregation of separate parts appropriately mated or joined together, e.g., carriers, assay substrates, coverslips, and/or spacers. Typically, the flow cells described herein will comprise one or more top components, one or more bottom components, where the one or more top components and bottom components structurally interact to form an interior portion used for biochemical analysis. In some aspects, the bottom component comprises a carrier that is substantially planar in structure, typically having a substantially flat lower surface and a substantially planar upper surface with a region that can receive an assay substrate.

As will be described in detail infra, there are two general configurations of flow cell carrier designs that may be implemented: top-side carrier designs and bottom-side carrier designs, depending upon source of illumination imaging. The designations refer to the location of the detection, e.g., illumination or imaging, with respect to the flow cell. In the case of a top-side carrier, the detection will occur from the top and the detection apparatus, e.g., an optical imager, is mounted above the assay substrate. In the case of a bottom-side carrier, the detection will occur from the bottom of the flow cell and the detection apparatus is mounted below the assay substrate. Although not described in great detail, it will be apparent to one skilled in the art upon reading the present disclosure that a top-side or bottom-side configuration can be used in a plane other than the horizontal plane—e.g., vertical mounting of the flow cell with detection effectively from the side of the flow cell—so the terms “top” and “bottom” should not be limited to the horizontal plane, but rather refer to the area of detection with respect to the flow cell itself.

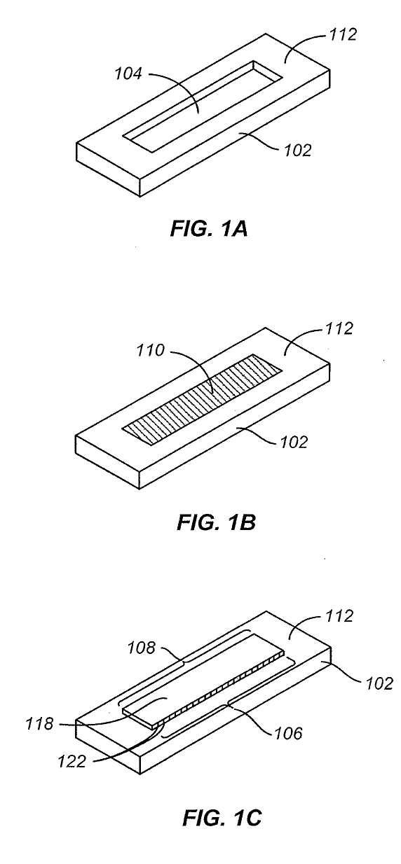

FIG. 1A is an isometric view of one aspect of a carrier 102 according to the claimed invention. A carrier 102 has a recessed region 104 to accommodate an assay substrate. FIG. 1B is an isometric view of one aspect of a carrier 102 showing the assay substrate 110 placed within the recessed region 104 so that the top surface of the substrate 110 is flush with the top surface of the carrier 112. To assure that the assay substrate is held tight against the carrier, holes may be fashioned into the carrier beneath where the assay substrate is provided so that a vacuum may be applied through a chuck upon which the carrier is mounted or via vacuum ports created within the carrier. FIG. 1C is an isometric view of the carrier and substrate with a coverslip 118 positioned above the substrate and carrier via attachment means. The coverslip 118 is positioned over the substrate using sealers 122 so that chambers are created between the bottom surface of the coverslip 118 and the top surface of the assay substrate 110. The positioning of the coverslide on the carrier also creates input 106 and output 108 ports compatible with biochemical processing and imaging.

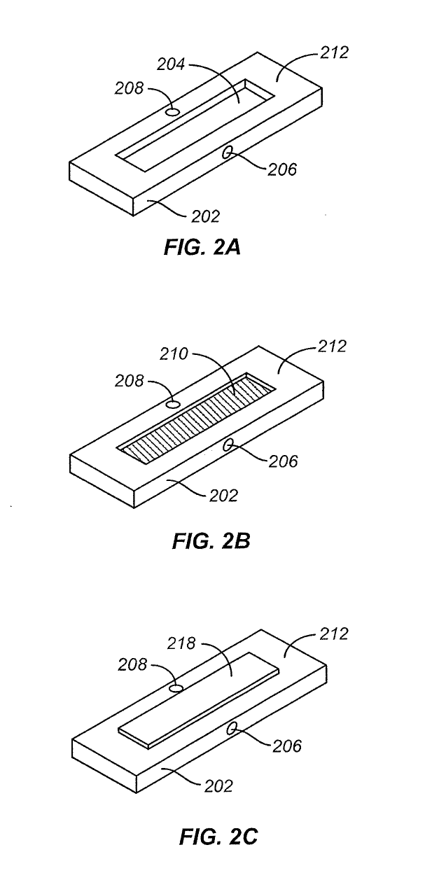

FIG. 2A is an isometric view of another aspect of a carrier 202 according to the claimed invention. Carrier 202 has a recessed region 204 to accommodate an assay substrate.

In addition, in this aspect, input 206 and output ports 208 are provided in the sides of the carrier that provide an inlet and outlet, respectively, to the recessed region 204. Although the figure illustrates one input and one output port, multiple ports can be provided along the length of the carrier to enhance delivery and evacuation. FIG. 2B is an isometric view of one aspect of a carrier 202 showing the top side of an assay substrate 210 placed in the recessed region 204. The top surface of the substrate is recessed with respect to the top surface of the carrier 212. The assay substrate may be attached to the carrier with, e.g., clips, bonded with glue, held in place by the coverslip or any other means compatible with biochemical processing and imaging. As with the previous aspect, holes may be fashioned into the carrier beneath where the assay substrate is provided so that a vacuum may be applied through a chuck upon which the carrier is mounted or via vacuum ports created within the carrier. The input 206 and output 208 ports are provided above the substrate surface in the recessed region to allow the introduction and exit of fluids to the surface of the substrate 210. FIG. 2C is an isometric view of the carrier with the coverslip 218 provided above the top surface of the assay substrate 210 to completely cover the assay substrate and attached to the top surface 212 of the carrier 202.

As seen in FIGS. 1C and 2C, a coverslip is mounted over the assay substrate. The face of the coverslip will be larger in area than the assay substrate so that all or substantially all of the top surface of the assay substrate is usable. Input and output ports are available at, e.g., both ends of the carrier so that reagents may be dispensed into the carrier and evacuated out of the carrier. The input port may be configured to have a small inlet feature to minimize surface area of the exposed liquid so as to minimize evaporation. In more involved implementations, valves or other fluid dispensing/plumbing-type fixtures may be fabricated at the input port locations.

The coverslip may be mounted to both the assay substrate and the carrier, which can be an advantageous configuration as this eliminates the need for the coverslip to be a mechanically robust structure in its own right. In some prior art implementations, the assay substrate was transferred by applying an end effector to the coverslip, which proved problematic as a coverslip typically is only 170 μM thick. By bonding the coverslip directly to the carrier, more surface area is available for bonding the coverslip without reducing the substrate surface, enabling a more robust flow cell with a further gain of functional area on the assay substrate.

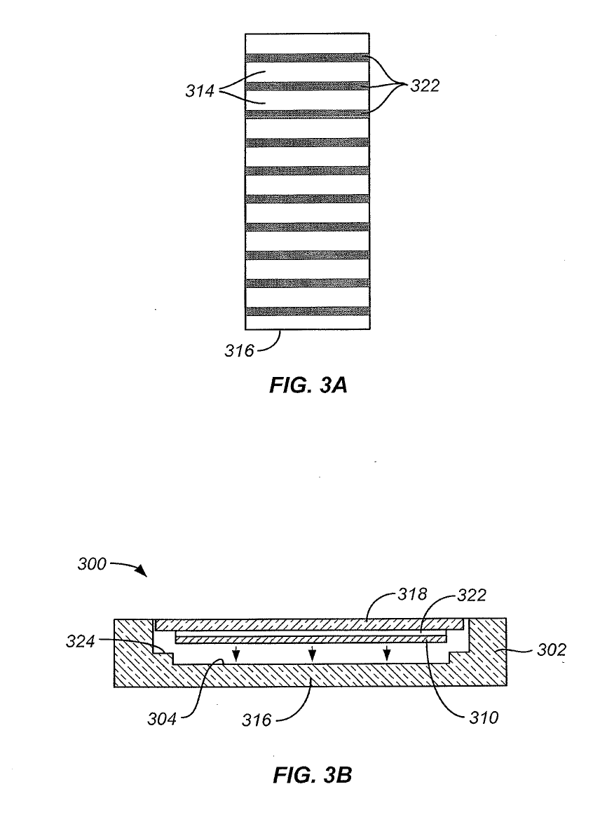

FIG. 3A is a top plan view of one aspect of a flow cell comprising an assay substrate 310, comprising spacers 322 and assay regions 314. Here, the spacers 322 and assay regions 314 are shown as parallel elements running lengthwise down the narrow dimension of the assay substrate; however, this configuration is exemplary only. Any configuration appropriate to the assay being run may be used including those implementing microfluidic valves and channels, as well as assay substrates employing microelectronics. FIG. 3B is a cross sectional view of a flow cell 300 comprising an assay substrate 310, carrier 302 and coverslip 318 with the cross section taken from one end 316 of the assay substrate 310. A spacer can be seen at 322, as well as a region 304 in the carrier 302 to accommodate the assay substrate 310, and a region 324 in the carrier 302 to accommodate the coverslip 318. The coverslip 318 is larger than the assay substrate 310 in at least one dimension (length or width), and in some aspects the coverslip 318 is larger than the assay substrate 310 in both length and width.

The carrier may comprise a variety of materials. The carrier material is selected generally for its compatibility with the full range of conditions to which the flow cells may be exposed, including extremes of pH, temperature, salt concentration, application of electric fields or use of optics. In some aspects, the carrier may comprise metals, ceramics, glass or polymeric materials. In other aspects, the carrier material comprises a material through which the assay substrate may be optically imaged, such as, e.g., silica-based substrates such as glass, quartz, silicon or polysilicon, as well as other transparent materials, such as transparent polymers, plastics and the like.

The assay substrate also can comprise a variety of materials, depending primarily, like the materials that comprise the carrier, on compatibility with the conditions to which the assay substrates will be exposed and the type and configuration of the imaging and optics employed. Typically, the assay substrate comprises silica-based substrates such as glass, quartz, silicon or polysilicon. Similarly, the materials chosen for the coverslip will take into account the conditions to which the coverslip will be exposed and the type and configuration of the imaging and optics employed, and will often comprise glass, quartz, silicon or polysilicon. In some aspects, particularly when used in assays that employ repeated steps of processing and imaging, the components of the flow cell are selected so as to provide high durability and reproducibility particularly relating to imaging. The coverslip is optionally coated with a hydrophobic material (e.g., by fluorination) to eliminate binding of biological materials to the coverslip surface.

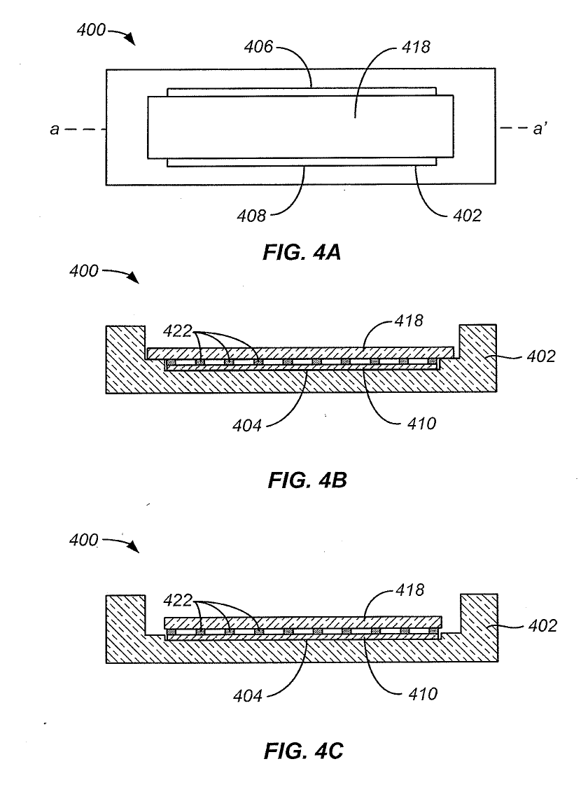

FIG. 4A is a top plan view of a flow cell 400 showing a carrier 402 and a coverslip 418. In this configuration, the input and output ports are indentations or recesses provided along the horizontal length of the flow cell for entry and exit of fluids in the channels which run in parallel fashion along the width of the flow cell. Input ports 406 and output ports 408 are here provided for each channel along the length of the slide. FIG. 4B is a cross-sectional view of the flow cell 400 of FIG. 4A taken along line a-a′, comprising a carrier 402, an assay substrate 410, spacers 422, a coverslip 418, a region 404 in the carrier 402 to accommodate the assay substrate 410, and a region 404 in the carrier 402 to accommodate the coverslip 418. FIG. 4C is a cross-sectional view of yet another aspect of a flow cell 400 according to the claimed invention. Again, a carrier 402, an assay substrate 410, spacers 422, a coverslip 418, a region 404 in the carrier 402 to accommodate the assay substrate 410, and a region 404 in the carrier 402 to accommodate the coverslip 418 are all seen. However, the configuration of FIG. 4C differs from FIGS. 4B as there is no specific region for the coverslip 318 provided by the carrier 402.

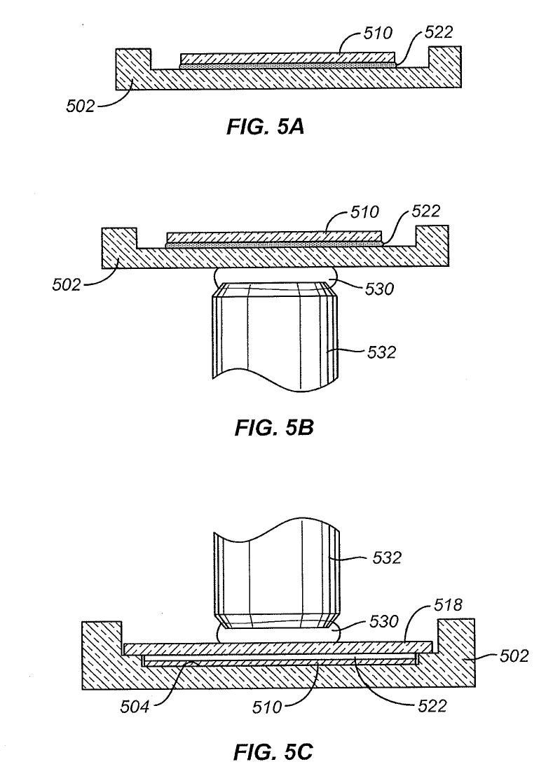

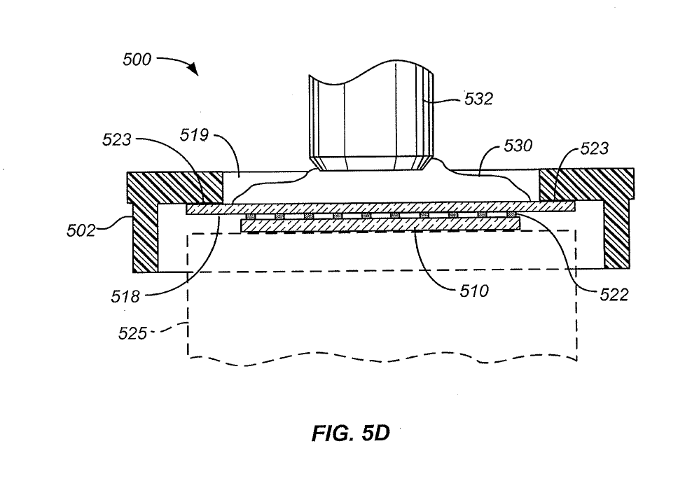

FIGS. 5A and 5B are side plan views of bottom-side implementations of the flow cell in use with a microscope objective. FIG. 5A shows a transparent carrier 502, which is also effectively the coverslide, and an assay substrate 510 provided on the carrier with a spacer 522. In FIG. 5B, the schematic in FIG. 5A is shown with an imaging detection device, here a microscope. The microscope objective 532 is placed beneath the transparent carrier 502 and immersion fluid 530 (e.g., water or oil) is placed between the objective 532 and the carrier 502 for detection. In contrast, FIG. 5C shows imaging of a top-side configuration. This flow cell comprises a carrier 502, which need not be transparent, an assay substrate 510, spacers 522, a coverslip 518, and a region 504 in the carrier 502 to accommodate the assay substrate 510. In bottom-side implementations such as those shown in FIGS. 5A and 5B, the carrier is generally made of an optically-quality material such as glass, quartz, plastic or the like. The microscope objective (or other imaging hardware) is mounted beneath the assay substrate and imaging is done through the carrier. The assay substrate is flipped upside down so that the functionalized surface of the assay substrate is adjacent the carrier (albeit separated by reagent buffer in many aspects). The immersion microscope objective 532 is adjacent the coverslip 518 and embedded in the immersion fluid bead 530.

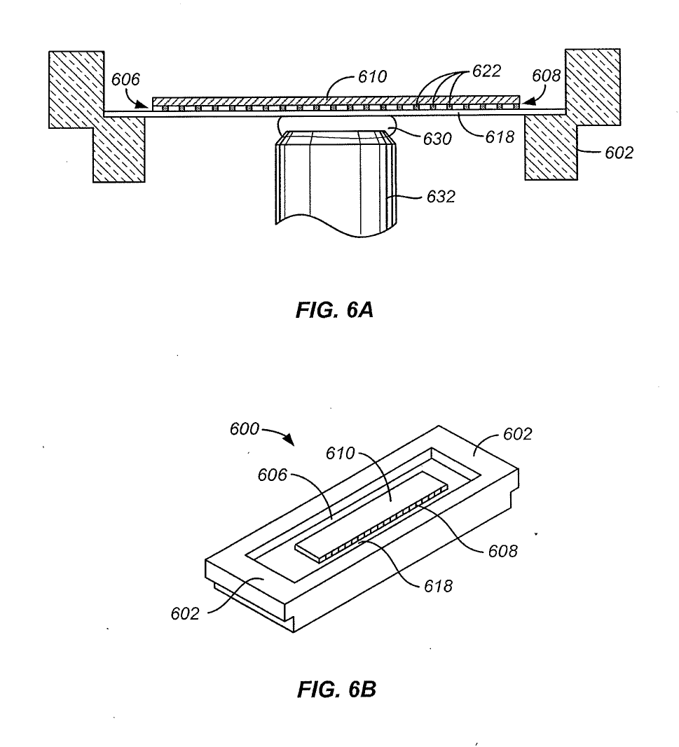

FIG. 5D shows imaging of a preferred top-side configuration of a flow cell 500 whereby a much more efficient use of assay substrate is achieved. This flow cell comprises a carrier 502, which need not be transparent and may be of molded plastic, an assay substrate 510, preferably of optical quality silicon or quartz, spacers 522 typically formed of glue lines whereby separate reaction chambers are defined on the substrate 510 and which have inlet ports for pipette injections and outlets ports for vacuum removal of reagents and other fluids, a coverslip 518, typically a thin glass slide adapted for use as an interface for immersion optics and mounted on all its edges 523 to the carrier 502 to form a sealed basin 519 above the assay substrate 510. In top-side implementations, the objective 532 is immersed in optical immersion fluid 530 whereby the reageant region between spacers 522 is scanned and imaged. The substrate 510 in turn rests directly on a flat surface such as vacuum chuck 525 to hold the substrate stable during imaging. This is similar to the bottom-side implementation of FIG. 6A, as hereinafter explained. In bottom-side implementations such as those shown in FIGS. 5A and 5B, the carrier is generally made of an optically-available material such as glass, silicon, plastic or the like. The microscope objective 532 (or other imaging hardware) is mounted beneath the assay substrate 510 and imaging is done through the carrier 502. The assay substrate 510 is flipped upside down so that the functionalized surface of the assay substrate is adjacent the carrier (albeit separated by reagent buffer in many aspects).

In a specific bottom-side implementation, illustrated in side view in FIG. 6A and top view in FIG. 6B, the coverslip 618 is a thin, optically-transmissive material such as glass, silicon, plastic or the like positioned in a separate carrier 602 in a manner that will not disrupt visualization of sample through the coverslip 618. The microscope objective 632 (or other imaging hardware) is mounted beneath the coverslip 618. The assay substrate 610 is placed over the coverslip 618 and separated from the coverslip 618 using spacers 622. Thus, the assay substrate 610 is flipped upside down so that the functionalized surface of the assay substrate is adjacent to the coverslip 618 (albeit separated by reagent buffer in many aspects). Imaging takes place through the coverslip 618, with the carrier 602 positioned on the edges of the coverslip 618 in a manner which provides stability of the flow cell 600 but which does not obstruct visualization of samples on the assay substrate 610 through the coverslip 618. The microscope objective 632 is placed beneath the coverslip 618, and immersion fluid 630 is placed between the objective 632 and the coverslip 618 for detection. The carrier may also be placed to provide inlet ports 606 (front end) and outlet ports 608 (back end of substrate 610) that are created by the placement of the carrier 602, the coverslip 618 and the assay substrate 610.

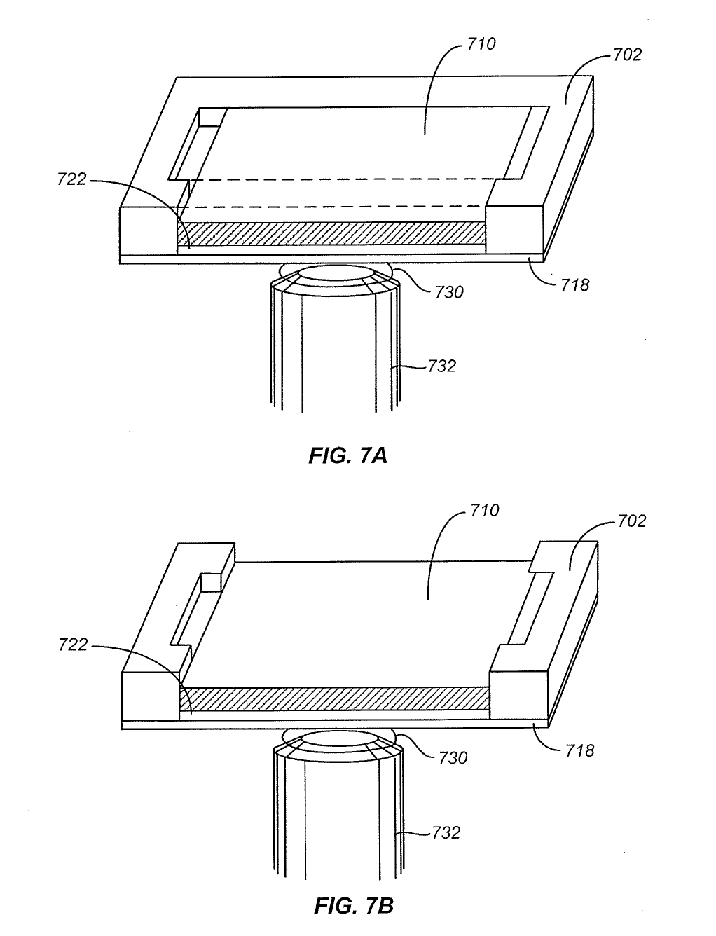

FIGS. 7A and 7B illustrate yet another bottom-side configuration that comprise a carrier 702 with a coverslip directly bound both to the carrier and to the assay substrate. The coverslip is preferably bound to the underside of the edges of the carrier, and the assay substrate bound directly to inside edges of the carrier. The microscope objective 732 (or other imaging hardware) is mounted beneath the coverslip, the assay substrate 710 is placed over the coverslip 718 and separated from the coverslip 718 using spacers 722. Imaging takes place through the coverslip 718, with the carrier 702 positioned on the edges of the coverslip 718 in a manner which provides stability of the flow cell 700 but which does not obstruct visualization of samples on the assay substrate 710 through the coverslip 718. In FIG. 7A, the carrier 702 surrounds the assay substrate 710, which is bound to the carrier 702 on all sides. A cutout view on the front of the figure illustrates the placement of the array substrate 710 with respect to the carrier 702. In 7B, the carrier 702 is bound to the assay substrate 710 on two opposite sides, with the spacers 22 forming a barrier on the edges of the assay substrate 710 not directly attached to the carrier 702. The microscope objective 732 is placed beneath the coverslip 718 and immersion fluid 730 is placed between the objective 732 and the coverslip 718 for detection.

Bottom-side implementations have the advantage that the microscope is mounted below the assay substrate, and it is often easier to stabilize the optical system, leading to simplification of the mechanical design of the optical system. An additional advantage to the bottom-side design is that there is no gap between the assay substrate and the carrier other than the chamber that is formed where the reagent buffer is contained. The bottom-side design also creates a natural interface between the reagents and the immersion fluid. When possible, it is preferable to use water as the immersion fluid because the index of refraction of water does not change as it evaporates. In addition, most conventional optics systems are designed for use with water. However, top-side implementations have the advantages of permitting the translation stages to be mounted to rigid surfaces underneath the carrier, there is no need for a hole for the objective and there is no need to cantilever the carrier.

Certain bottom-side carrier designs may have limitations in the use of analyzing specific molecules, e.g., spherical aberrations may result due to imaging through a thick carrier. For example, high numerical aperture microscope objectives are usually designed to accommodate a coverslip of 150 to 170 μm. However, potential aberrations may be addressed by configuring the carrier in various ways (for example, those described infra) or by providing a carrier with a thickness of about 300 μm or so (appropriate if the carrier comprises fused silica (n=1.46)). Alternatively, optimized carriers may be constructed from materials with lower indices of refraction. In addition, if warping of the flow cell structure is potentially problematic, a real-time laser focus system may be employed to compensate for such warping and to ensure proper detection and analysis.

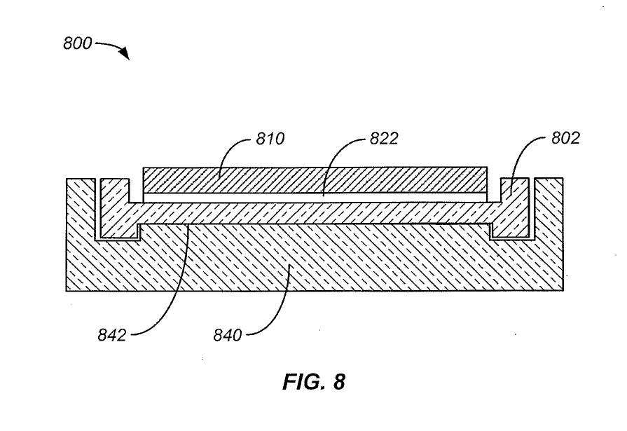

FIG. 8 shows a flow cell 800 according to yet another aspect of the claimed invention, showing a carrier 802, an assay substrate 810, and a spacer 822 between the assay substrate 810 and the carrier 802. In FIG. 8, the flow cell 700 further comprises a carrier cassette 840 that supports the carrier 802 through a region 842 portion of the carrier cassette 840. As described, in some implementations, particularly those employing bottom-side imaging, the portion of the carrier supporting the assay substrate may be configured to be thin to optimize optical imaging. In such implementations, a carrier cassette may be used to further support the carrier during processing that does not involve optical imaging.

The input port or ports allow the introduction into the substantially sealed chamber of fluids needed to process the sample on the support. Typically such fluids will be buffers, solvents (e.g. ethanol/methanol, xylene), reagents (e.g., primer- or probe-containing solutions) and the like. The output port allows for the processing fluids to be removed from the sample (e.g., for washing, or to allow the addition of a further reagent). Preferably, the orientation is such that the input port is directly opposite the output port in the flow cell, although other configurations can be envisioned where the output port could be positioned so that multiple channels could use a single output port or empty into a specific area, e.g., those on the edge of the flow cell, could have an output port position in the side of the channel rather than the end opposite the input port.

A number of arrangements for appropriate fluid delivery means can be envisaged. In a preferred embodiment a number of reservoirs of processing fluids, (e.g., buffers, stains, etc.) are provided, each reservoir being attached to a pumping mechanism. Preferred pumping mechanisms include, but are not limited to syringe pumps, such as those manufactured by Hook and Tucker, (Croydon, Surrey, UK), or Kloen having a stroke volume of between 1 and 10 ml. One such pump may be provided for each processing fluid reservoir, or a single pump may be provided to pump fluid from each a plurality of reservoirs, by means of a multi-port valve configuration.

Each syringe pump can in turn be attached to a central manifold (such as a universal connector). Preferably the central manifold feeds into a selective multi-outlet valve such that, if desired, where a plurality of samples are being processed simultaneously, each sample may be treated with a different processing fluid or combination of processing fluids. A suitable selective multi-outlet valve is a rotary valve, such as the 10 outlet rotary valve supplied by Omnifit (Cambridge, UK). Thus each outlet from the multi-outlet valve may be connected to a separate flow cell. One or more filters may be incorporated if desired. Typically a filter will be positioned between each reservoir and its associated syringe pump.

Each syringe pump may be actuated individually by the computer control means, or two or more pumps may be actuated simultaneously to provide a mixture of two or more processing fluids. Controlling the rate of operation of each pump will thus control the composition of the resulting mixture of processing fluids.

In an alternative embodiment, the fluid delivery means comprises two or more piston/HPLC-type pumps, each pump being supplied, via a multi-inlet valve, by a plurality of processing fluid reservoirs. Suitable pumps are available, for example, from Anachem (Luton, Beds, UK). The multi-inlet valve will be a rotary valve. Each pump will feed into a rotary mixer, of the type well known to those skilled in the art, thus allowing variable composition mixtures of processing fluids to be produced, if desired.

In certain aspects, the processing fluid or mixture of processing fluids is then passed through an in-line filter and then passes through a selective multi-valve outlet (such as a rotary valve) before being fed into the flow cells.

As an alternative to the generally “parallel” supply of processing fluids defined above, the processing fluids may be supplied in “series” such that, for example, fluid is passed from one substantially sealed chamber to another. This embodiment has the advantage that the amount of reagent required is minimized.

In aspects of the invention comprising one or more valves, typically the valve will be a three-way valve with two inlets, and one outlet leading to the substantially sealed flow cell. One of the valve inlets is fed, indirectly, by the reservoirs of processing fluid. The second inlet is fed by a local reservoir which, typically, will be a syringe, pipette or micro-pipette (generally 100-5000 μl volume). This local reservoir may be controlled by the computer control means or may be manually controlled. The local reservoir will typically be used where a reagent is scarce or expensive. The provision of such a local reservoir minimizes the amount of reagent required, simplifies cleaning, and provides extra flexibility in that each flow cell may be processed individually, if required.

In a specific aspect of certain embodiments of the invention, the “flow” for use in the flow cell reaction is achieved by gravity force, e.g., placement of the flow cell at an angle or by the use of an absorbent material applied on the out-put edge of the flow cell. In other aspects of the embodiments, the flow is produced using either mechanical or electrical means, e.g., the introduction of a vacuum apparatus to the out-put edge of the flow cell. The flow cell in such embodiments may be substantially sealed, or may have the flow directed by forces applied to the input port and/or output port for transfer of fluids through the flow cell.

In a preferred aspect, fluid enters the flow cell from the top and is carried through the reaction via gravity, exiting the flow cell via a output port at the bottom. The output port can empty into a common collecting duct, which duct drains into a collecting vessel. The vessel is desirably removable from the apparatus to allow for periodic emptying and/or cleaning.

The invention further relates to manufacture of and use of the flow cell and/or the apparatus of the invention in processing a sample on a support, such that the invention provides: a method of processing a sample using a flow cell; a method of making a flow cell; and a method of constructing an analysis system using one or more flow cells in accordance with the present invention.

### Flow Cell Sealing Means

The channels within a single flow cell may be provided using a sealer (which may serve both a spacing and a separating function) which may comprise materials that, when sandwiched between the assay substrate and the coverslip, provide a water-tight seal between assay channels, provide a water-tight perimeter around the assay substrate, and/or provide a structural function to give height to assay channel (i.e., in essence providing chambers for the assay regions). As with the other components of the flow cell, the conditions to which the spacers will be exposed and the type and configuration of the imaging and optics employed in the assay are taken into consideration when choosing the materials from which the spacers are formed. Materials contemplated for use as spacers include double-sided substrate adhesives (i.e., tape), wire, rubberized or polymer-coated wire, glues and adhesives, polymeric strips and the like.

It should be understood that the configuration of the spacers, or any other supports of the microfluidic channels and/or electronics may be adapted to suit the particular biochemical assay being performed, and to accommodate several input ports and/or output ports as may be required. Thus, in certain aspects, it is generally preferred that the flow cell comprises sealing means to assist in the formation of a substantially sealed chamber with multiple reaction channels/chambers. The sealing means may be an integral part of the support retaining member, or may be provided as a separate component of the flow cell. In aspects in which a specific distance between the substrate and the coverslip is desired, this can be obtained using the sealing means of the flow cell.

In another aspect of the invention using small volumes in the analytic reactions, the flow cell components are directly connected via the use of an adhesive. The adhesive is preferably introduced to a surface that provides optimal adhesion between the various flow cell components, e.g., a slide comprising an array and a coverslip. The adhesive may be a solid, such as a tape, or may be an adhesive applied as a liquid or gel that can subsequently be dried or cured into a solid form. The solid adhesive provides height to the reaction chamber by virtue of its thickness. A liquid or gel can also contain solid or semi-solid particles of a specific size (e.g., glass or plastic beads) that will remain a particular thickness when the liquid or gel adhesive dries, thus defining the height of the reaction chamber.

In certain aspects, the sealing means comprises a gasket, which may be made of silicon rubber or other suitable material. In a specific aspect, the sealing means comprises an O-ring gasket, the shape of which is generally that of a frame-like surround provided in a groove in one portion of the support retaining member. In an alternative embodiment the sealing means comprises a flattened frame-like surround gasket (about 50 to 150 μm thick). In other specific aspects, a gasket or other spacer material can be attached with an adhesive.

Either type of gasket may be discarded after a single use (if, for example, contaminated with a radioactive probe) or may be re-used if desired. The flattened gasket embodiment is particularly suitable as a disposable gasket, to be discarded after a single use. It will be apparent that the thickness of the gasket (which can be readily altered by exchanging gaskets) may, in part, determine the volume of the substantially sealed chamber.

Typically, where the nucleic acid sample is supported on a slide, the substantially sealed chamber will have a volume of between 30 μl and 300 μl, preferably between 50-150 μl. This small volume allows for economical use of reagents and (where temperature regulation is involved) a rapid thermal response time. Where a larger molecule is provided for analysis, or where larger volumes of sample are used in the analysis, the chamber will generally be larger (up to 2-3 mls).

### Assays

Technology is described herein for providing improved flow cells that may be used as part of an overall system for biological assays. In preferred aspects, the flow cells of the claimed invention are used for polynucleotide analysis including, but not limited to, expression and transcriptome analysis using nucleic acid microarrays, PCR and other polynucleotide amplification reactions, SNP analysis, proteome analysis, and the like, and particularly nucleic acid sequencing. The following filed patent applications provide additional information on various assays that may be used in conjunction with the flow cells and flow cell components described herein: U.S. patent application Ser. No. 11/451,691 filed Jun. 13, 2006; Ser. No. 11/679,124 filed Feb. 24, 2007; 60/991,141, filed Nov. 29, 2007 and 60/991,605, filed Nov. 30, 2007; and in various systems such as those described in U.S. Pat. App. 60/983,886 filed Oct. 30, 2007, and its counterpart application Ser. No. 12/261,548 file Oct. 30, 2008.

In particular aspects, the flow cell is adapted so as to be suitable for use in performing replication and/or amplification (e.g., circle dependent replication, circle dependent amplification or polymerase chain reaction amplification) on samples attached to a support. In such an embodiment, the flow cell must have an opening to allow the addition of further reagents. This opening must be designed so that it is transitory and the flow of any new liquids is very tightly controlled to prevent any leakage from the flow cell and to prevent contamination of the flow cell upon addition of any new reagents.

In a particular aspects of certain embodiments, for example those envisaged for use with PCR or other reactions in which tightly controlled temperature regulation is required, the flow cell is equipped with temperature control means to allow for rapid heating and cooling of the sample and PCR mix (i.e. thermal cycling). Typically the flow cell will be provided with an electrical heating element or a Peltier device. The flow cell may also be adapted (e.g., by provision of cooling means) to provide for improved air cooling. Temperature control in the range 3°-105° C. is sufficient for most applications.

### Use in Sequencing Reactions

The flow cells of the present disclosure are useful in numerous methods for biochemical interrogation of nucleic acids of unknown sequence. For example, flow cells of the invention can be used with hybridization-based methods, such as disclosed in U.S. Pat. Nos. 6,864,052; 6,309,824; and 6,401,267 and U.S. Published Patent Application 2005/0191656, which are incorporated by reference; sequencing by synthesis methods, such as disclosed in U.S. Pat. Nos. 6,210,891 6,828,100; 6,833,246; 6,911,345; Ronaghi et al (1998), Science, 281: 363-365; and Li et al, Proc. Natl. Acad. Sci., 100: 414-419 (2003), which are incorporated by reference; and ligation-based methods, e.g., WO1999019341, WO2005082098, WO2006073504 and Shendure et al (2005), Science, 309: 1728-1739, which are incorporated by reference.

In particular aspects, multiple flow cells are used in high throughput analysis with multiple biochemical sequencing reactions. The flow cells may, for example, be arranged side-by-side, or one in front of the other in a sequencing reaction system. The multiple flow cells optionally includes nucleic acids or primers attached to the substrate of the flow cell, either randomly or in a predetermined manner, so that the identity of each nucleic acid in the multiple flow cells can be monitored throughout the reaction processes. The nucleic acids or primers can be attached to the surface such that at least a portion of the nucleic acids or primers are individually optically resolvable.

In one preferred aspect of the embodiments, the flow cells for use in systems of the invention comprise a substrate on which nucleic acids of unknown sequence are immobilized.

In certain aspects of the embodiments of the invention, a clamping means is capable of clamping together a plurality of flow cells. Typically, from one to around twelve or sixteen flow cells may be clamped simultaneously by a single clamping means. The flow cells can be arranged in the clamping means in a substantially horizontal or substantially vertical manner, although any position intermediate between these two positions may be possible.

The present specification provides a complete description of the methodologies, systems and/or structures and uses thereof in example aspects of the presently-described technology. Although various aspects of this technology have been described above with a certain degree of particularity, or with reference to one or more individual aspects, those skilled in the art could make numerous alterations to the disclosed aspects without departing from the spirit or scope of the technology hereof. Since many aspects can be made without departing from the spirit and scope of the presently described technology, the appropriate scope resides in the claims hereinafter appended. Other aspects are therefore contemplated. Furthermore, it should be understood that any operations may be performed in any order, unless explicitly claimed otherwise or a specific order is inherently necessitated by the claim language. It is intended that all matter contained in the above description and shown in the accompanying drawings shall be interpreted as illustrative only of particular aspects and are not limiting to the embodiments shown. Changes in detail or structure may be made without departing from the basic elements of the present technology as defined in the following claims. In the claims of any corresponding utility application, unless the term “means” is used, none of the features or elements recited therein should be construed as means-plus-function limitations pursuant to 35 U.S.C. §112, ¶6.

What is claimed is:

1. A biochemical analysis system comprising:
(a) an assay flow cell, wherein said assay flow cell comprises:
an optically transmissive coverslip having an observation surface;
a planar assay substrate disposed upon and within boundaries of said coverslip such that all assay regions of the assay substrate are observable through said coverslip; and
a carrier, said carrier forming a frame operative to protect, and to provide a mechanical transport for, said coverslip and said assay substrate;
said assay substrate comprising at least one assay region having a fluid inlet and a fluid outlet isolated from said observation surface; and
(b) a detection device disposed to observe said assay regions.

2. The biochemical analysis system according to claim 1 wherein spacers are provided as support structure and isolation barriers between said assay substrate and said coverslip to form a plurality of said assay regions.

3. The biochemical analysis system according to claim 2 wherein said spacers are formed by glue lines across said assay substrate.

4. The biochemical analysis system according to claim 2 wherein each said assay region has a corresponding fluid inlet and a fluid outlet for permitting flow of reactants through the assay region.

5. The biochemical analysis system according to claim 1 wherein said coverslip is disposed in a recess of said carrier forming a basin sealed along edges of said coverslip.

6. The biochemical analysis system according to claim 1 wherein the carrier has a top surface in which is a recess, and wherein said assay substrate is disposed within said recessed region and under the coverslip opposing said observation surface.

7. The biochemical analysis system according to claim according to claim 1 wherein the carrier has a top surface in which is a recess, and wherein said coverslip is disposed within said recessed region and sealed around its edges forming a basin for retaining immersion fluid on said observation surface of said coverslip in isolation from reagents and other fluids on said assay substrate.

8. The biochemical analysis system according to claim according to claim 7 wherein said assay substrate is unobstructed such that said assay substrate may be mounted upon a chuck.
